(19) United States
(12) Patent Application Publication
(10) Pub. No.: US 2019/0191984 A1
(43) Pub. Date: Jun. 27, 2019

(54) LAPAROSCOPIC SURGICAL METHOD

(71) Applicant: OLYMPUS CORPORATION, Tokyo (JP)

(72) Inventors: Asuka NAKAMURA, Tokyo (JP); Yohei TANIKAWA, Tokyo (JP); Satoshi TAKEKOSHI, Tokyo (JP)

(73) Assignee: OLYMPUS CORPORATION, Tokyo (JP)

(21) Appl. No.: 15/854,012

(22) Filed: Dec. 26, 2017

(51) Int. Cl. A61B 1/313; A61B 5/00

(52) U.S. Cl. CPC A61B 1/3132; A61B 1/0008; A61B 5/0084

(57) ABSTRACT

A laparoscopic surgical method including: observing an interior of an abdominal cavity by using a laparoscope and selecting an optical clearing site to be subjected to optically clearing of fat tissue that covers target tissue; allowing an optical clearing agent to act on the selected optical clearing site; checking visibility of the target tissue through the optical clearing site on which the optical clearing agent has been allowed to act; and treating the target tissue while observing, by using the laparoscope, the target tissue the visibility of which has been checked.

### TECHNICAL FIELD

The present invention relates to a laparoscopic surgical method, and relates, in particular, to a laparoscopic surgical method with which it is possible to assist a surgeon by improving the visibility of target tissue by clearing fat tissue by using an optical clearing agent.

### BACKGROUND ART

In laparoscopic surgery, unlike laparotomy, it is difficult to tactually check tissue by directly touching the tissue with the hand or to push the tissue aside. Therefore, in laparoscopic surgery, in the case in which target tissue, such as blood vessels, organs, or the like, is hidden by peripheral fat tissue, thus making it impossible to directly visually ascertain the target tissue, cautious work such as making an incision in or detaching the peripheral fat tissue, or the like is required in order to disclose the target tissue. Such work has been a burden on the surgeon. In the case of an obese patient having a large quantity of visceral fat, the difficulty of performing laparoscopic surgery is further increased due to the presence of thick fat tissue that covers the target tissue.

On the other hand, there have been advances in the development of optical clearing agents with which biological tissue is made transparent by decreasing light scattering (for example, see Patent Literature 1).

### CITATION LIST

### Patent Literature

{PTL 1} U.S. Patent Application Publication No. 2015/0351618

### DESCRIPTION

### Summary of Invention

An aspect of the present invention is a laparoscopic surgical method including: observing an interior of the abdominal cavity by using a laparoscope and selecting an optical clearing site to be subjected to optical-clearing in fat tissue that covers target tissue; allowing an optical clearing agent to act on the selected optical clearing site; checking visibility of the target tissue through the optical clearing site on which the optical clearing agent has been allowed to act; and treating the target tissue while observing, by using the laparoscope, the target tissue in which the visibility thereof has been checked.

### BRIEF DESCRIPTION OF DRAWINGS

FIG. 1 is a flowchart showing a laparoscopic surgical method according to an embodiment of the present invention.

FIG. 2 is a diagram for explaining an example of an optical clearing site in cholecystectomy.

FIG. 3A is a diagram for explaining a preferable injection position for an optical clearing agent.

FIG. 3B is a diagram for explaining another injection position for the optical clearing agent.

FIG. 4A is a diagram for explaining a method of injecting the optical clearing agent between a membrane and a fat-tissue surface.

FIG. 4B is a diagram for explaining the method of injecting the optical clearing agent between the membrane and the fat-tissue surface.

FIG. 4C is a diagram for explaining the method of injecting the optical clearing agent between the membrane and the fat-tissue surface.

FIG. 5A is a diagram for explaining an example of an injection pattern of the optical clearing agent.

FIG. 5B is a diagram for explaining a permeation area of the optical clearing agent in the injection pattern in FIG. 5A.

FIG. 6A is a diagram for explaining another example of the injection pattern of the optical clearing agent.

FIG. 6B is a diagram for explaining a permeation area of the optical clearing agent in the injection pattern in FIG. 6A.

FIG. 7 is a diagram for explaining a method of checking the visibility of target tissue.

### DESCRIPTION OF EMBODIMENT

A laparoscopic surgical method according to an embodiment of the present invention will be described below with reference to the drawings.

The laparoscopic surgical method according to this embodiment relates to a laparoscopic surgical method in which fat tissue covering target tissue is made transparent by using an optical clearing agent, thus making it possible to observe the target tissue through the fat tissue by using a laparoscope, and the target tissue is subsequently treated.

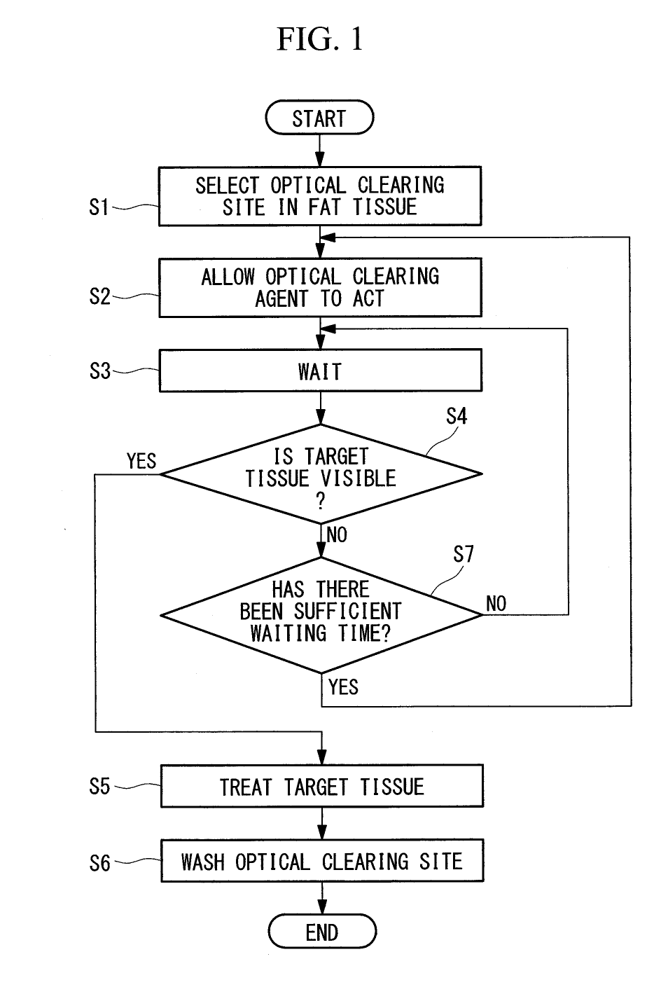

As shown in FIG. 1, the laparoscopic surgical method includes: a first step S1 of selecting, in the fat tissue that covers the target tissue in an abdominal cavity, an optical clearing site to be made clear; a second step S2 of allowing the optical clearing agent to act on the selected optical clearing site; a third step S3 of waiting until the optical clearing agent permeates the fat tissue; a fourth step S4 of checking the visibility of the target tissue via the optical clearing site; a fifth step S5 of treating the target tissue; and a sixth step S6 of washing the optical clearing site.

In the first step S1, the interior of the abdominal cavity is observed by using the laparoscope, and an optical clearing site on which the optical clearing agent is allowed to act is selected in the fat tissue that covers the target tissue. Because the fat tissue is optically opaque, it is not possible to observe, by using the laparoscope, the target tissue that is hidden behind the fat tissue with respect to the laparoscope. Therefore, a surgeon recognizes the position of the target tissue on the basis of an anatomical landmark (for example, tissue in the area surrounding the target tissue and a characteristic tissue structure), and selects, as the optical clearing site, a site in the fat tissue that is appropriate for observing the target tissue.

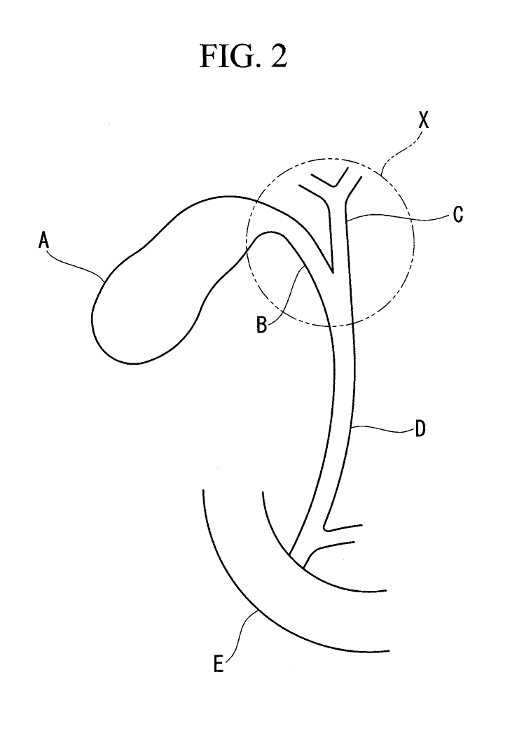

For example, as shown in FIG. 2, in the case of surgery for removing a gallbladder A, a cystic duct B and the cystic artery are cut. The cystic duct B and a common hepatic duct C are branched off from a common bile duct D, and, in order to correctly recognize and cut the cystic duct B, it is necessary to correctly ascertain the branched structure of the cystic duct B, the common hepatic duct C, and the common bile duct D. Therefore, a site that is appropriate for observing the cystic duct B and the branched structure is selected as an optical clearing site X. Reference sign E is the duodenum.

Next, in the second step S2, the optical clearing agent is applied to the optical clearing site X selected in the first step S1, and the optical clearing agent is allowed to act thereon. The optical clearing agent is, for example, a saccharide-compound aqueous solution and possesses the property of permeating the fat tissue and the property of decreasing light scattering. The optical clearing agent that has permeated the fat tissue exhibits an effect of making the fat tissue optically clear by decreasing light scattering in the fat tissue.

As the method of applying the optical clearing agent, a method in which the optical clearing agent is dripped or sprayed on the optical clearing site X or a method in which a member that is impregnated with the optical clearing agent is attached to the optical clearing site X could be employed. The method in which the optical clearing agent is dripped on, sprayed on, or attached to the fat tissue from outside is advantageous in that the procedures involved are easy.

As the method of applying the optical clearing agent, a method in which the optical clearing agent is injected on the inner side (fat-tissue side) of a membrane that covers a fat-tissue surface may be employed. The fat-tissue surface is covered with a thin membrane that is substantially optically clear. The optical clearing agent injected on the inner side of the membrane is retained on the inner side of the membrane for a certain period of time without flowing out to the outer side (the side facing away from the fat tissue), and gradually permeates the fat tissue toward deeper layers thereof. Therefore, it is possible to allow the optical clearing agent to efficiently act on the fat tissue.

The injection of the optical clearing agent is performed by disposing a distal-end portion of a needle on the inner side of the membrane by making the needle penetrate the membrane, and by discharging the optical clearing agent from an outlet of the distal-end portion of the needle on the inner side of the membrane.

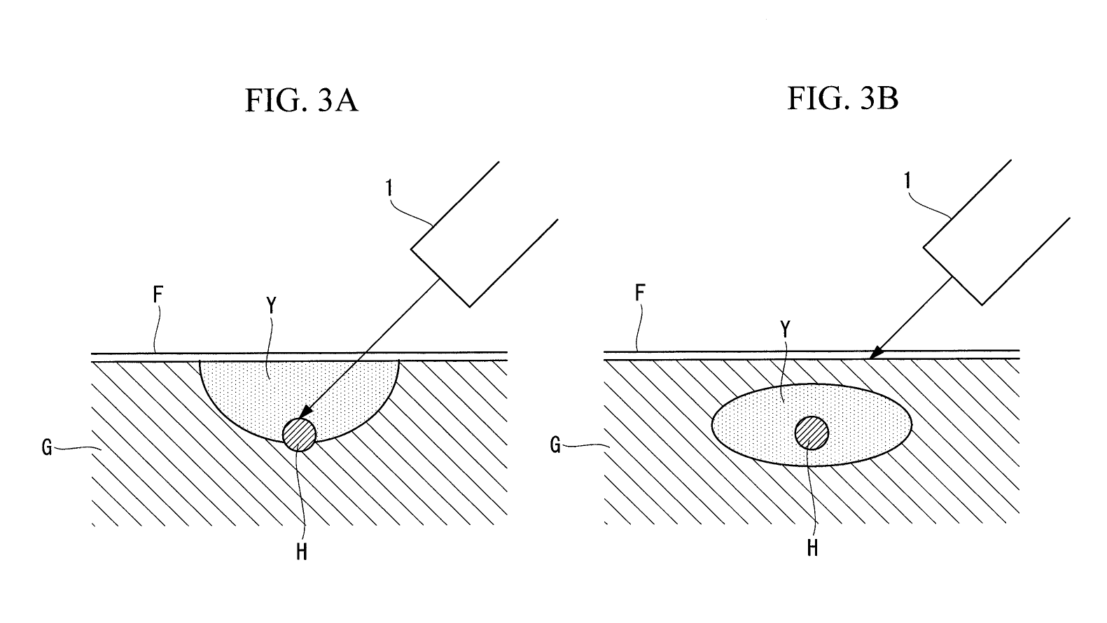

As shown in FIG. 3A, it is preferable that an injection position (piercing depth of the needle) of an optical clearing agent Y be between a membrane F and a surface of fat tissue G, or at a surface layer of the fat tissue G. By doing so, it is possible to allow the optical clearing agent Y to act on target tissue H in the depth direction from the surface of the fat tissue G, and it is possible to ensure, between a laparoscope 1 that is disposed on the outer side of the membrane F and the target tissue H, a satisfactorily optically clear path for observing the target tissue H by using the laparoscope 1. The target tissue H in FIGS. 3A and 3B is tubular tissue embedded in the fat tissue G, such as the cystic duct B, a blood vessel, or the like.

As shown in FIG. 3B, in the case in which the injection position of the optical clearing agent Y is at a relatively deep position in the fat tissue G, it is conceivable that a region in which the fat tissue G has not been made clear because the optical clearing agent Y has not acted thereon occurs in a portion of the fat tissue G that is closer to the surface thereof than the injection position is. When the opaque fat tissue G exists in a portion that is closer to the surface thereof than the fat tissue G that has been made clear is, it is not possible to achieve a satisfactory effect of visualizing the target tissue H through the fat tissue G.

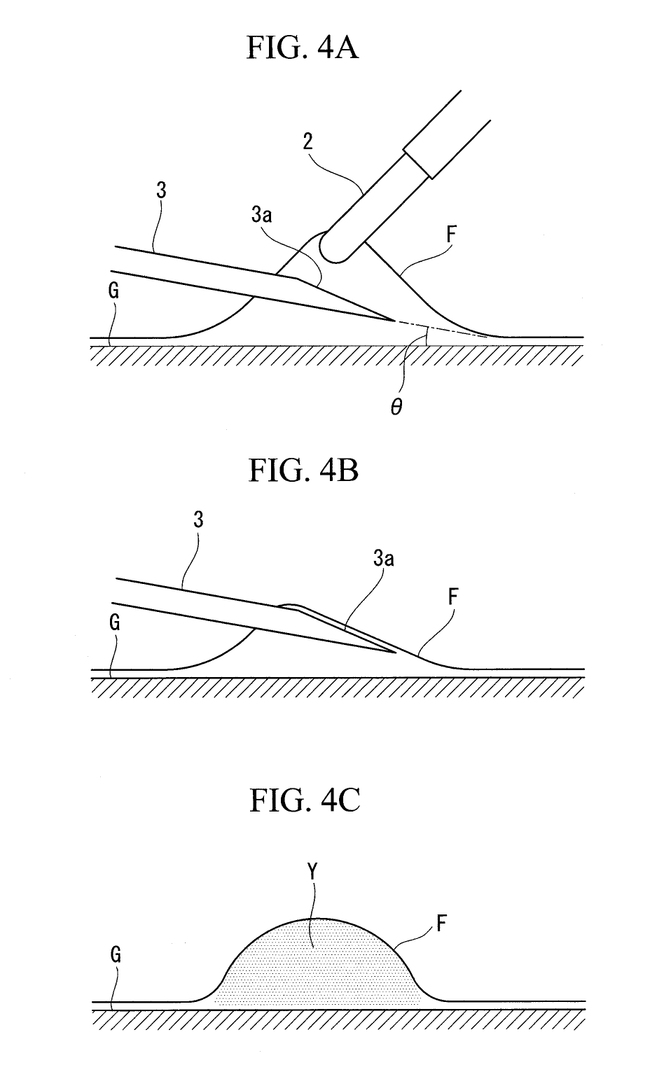

FIGS. 4A to 4C show specific methods of injecting the optical clearing agent Y.

First, as shown in FIG. 4A, by using forceps 2, the membrane F is held and lifted up in a direction in which the membrane F is separated from the surface of the fat tissue G. Next, a distal-end portion of a needle 3 is made to pierce through the lifted membrane F, thus being inserted between the membrane F and the surface of the fat tissue G. At this time, it is preferable that a piercing angle θ of the needle 3 with respect to the surface of the fat tissue G be 0° (in other words, parallel to the surface of the fat tissue G) to 45°. At the distal-end portion of the needle 3, a cut surface 3a which is inclined with respect to the longitudinal direction and in which an outlet opens is provided. It is preferable that, between the membrane F and the surface of the fat tissue G, the distal-end portion of the needle 3 be disposed so that the cut surface 3a faces the membrane F.

Next, as shown in FIG. 4B, an inner surface of the membrane F is brought into contact with the cut surface 3a of the needle 3 by relaxing the lifted membrane F, and the optical clearing agent Y is slowly discharged in this state from the outlet of the cut surface 3a. By being retained between the inner surface of the membrane F and the surface of the fat tissue G, the optical clearing agent Y discharged from the outlet causes the membrane F to swell up, as shown in FIG. 4C. In the case in which the injection speed is high, the optical clearing agent Y may leak out to the outer side of the membrane F from the hole in the membrane F formed by the needle 3. Subsequently, the injection of the optical clearing agent Y is completed by removing the needle 3 from the membrane F.

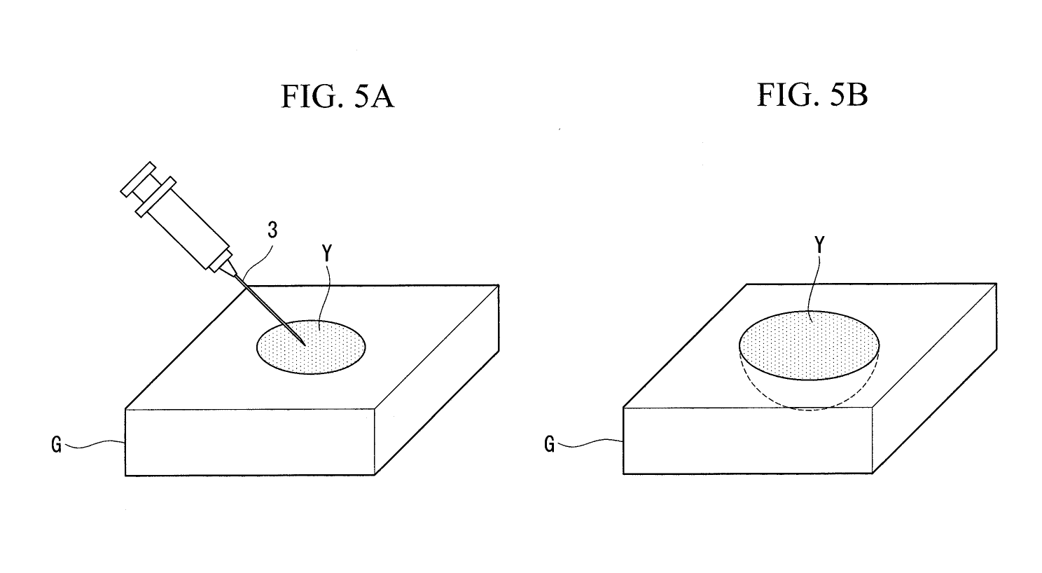

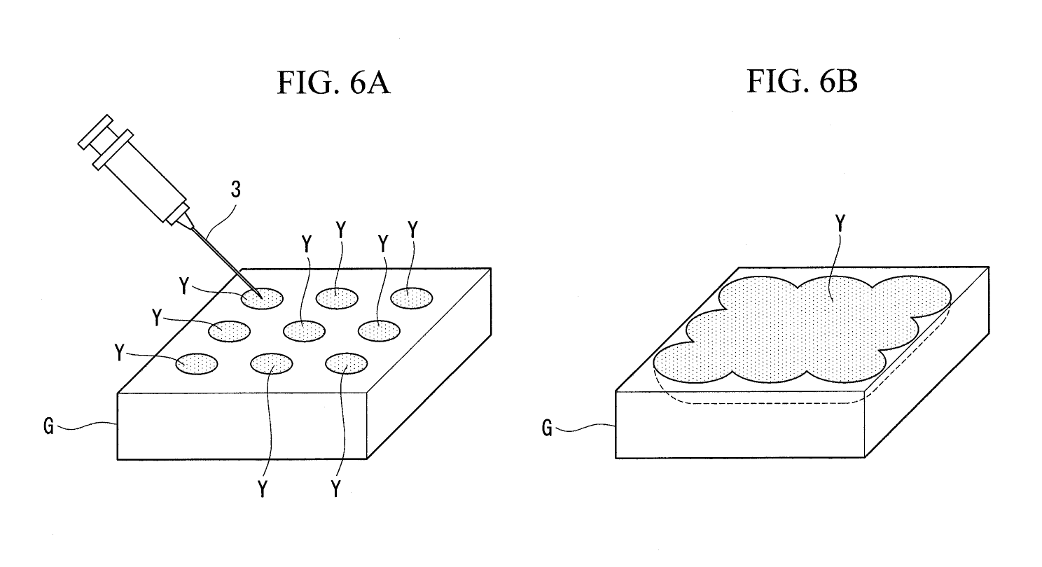

FIGS. 5A to 6B show examples of injection patterns of the optical clearing agent Y.

In the case in which a large amount of the optical clearing agent Y is injected in one spot, as shown in FIG. 5A, the permeation area of the optical clearing agent Y becomes small in a direction parallel to the surface of the fat tissue G and becomes large in the depth direction of the fat tissue G, as shown in FIG. 5B.

In the case in which a small amount of the optical clearing agent Y is injected at each of multiple points, as shown in FIG. 6A, the permeation area of the optical clearing agent Y becomes large in the direction parallel to the surface of the fat tissue G and becomes small in the depth direction of the fat tissue G, as shown in FIG. 6B.

After the optical clearing agent Y is applied to and allowed to act on the fat tissue G, in the subsequent third step S3, the surgeon waits until the optical clearing agent Y permeates the fat tissue G.

In the case in which the optical clearing agent Y is applied to the fat tissue G by means of injection, a rough standard of the waiting time is approximately several seconds to 30 minutes. The waiting time of approximately 10 minutes results in the greatest optical clearing effect of the optical clearing agent Y in the fat tissue G. When the waiting time exceeds 30 minutes, the optical clearing effect gradually decreases due to metabolism. When the waiting time exceeds 60 minutes, the optical clearing effect is almost entirely lost.

While waiting, the surgeon may prepare for treatment and washing to be performed in the subsequent steps S5 and S6. For example, in the case of cholecystectomy, preparations for detaching the gallbladder bed, injecting additional optical clearing agent Y, washing, and so forth are performed.

Next, in the fourth step S4, the fact that it is possible to visually ascertain the target tissue H, which exists behind an optical clearing site X with respect to the laparoscope 1, through the optical clearing site X is checked.

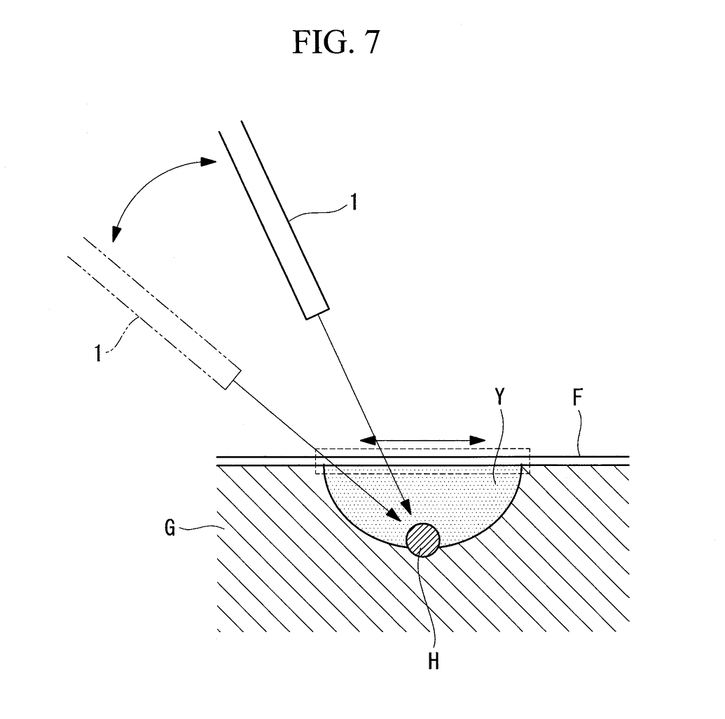

As shown in FIG. 7, the visibility of the target tissue H is checked, for example, by means of normal observation in which white illumination light is used by using the laparoscope 1. In the case in which the target tissue H is the blood vessel, the visibility may be checked by means of NBI (Narrow Band Imaging) observation in which blue and green narrow-band light beams are used. In the case in which the target tissue H is tubular tissue, such as the cystic duct B or the blood vessel, the visibility may be checked by observing fluorescence coming from a fluorescent agent administered to the target tissue H.

When checking the visibility of the target tissue H, the optical clearing site X may be shifted with respect to the target tissue H by pulling, by using a treatment tool such as the forceps 2, the membrane F and the surface layer of the fat tissue G (see the broken-line rectangle in FIG. 7) in the direction parallel to the surface of the fat tissue G.

In addition, the direction in which the target tissue H is observed may be a direction that is substantially perpendicular to the surface of the fat tissue G or a direction that is inclined with respect to the surface of the fat tissue G. By observing the target tissue H from an inclined direction, it is possible to ascertain the depth at which the target tissue H is located in the fat tissue G.

In the fourth step S4, in the case in which it is possible to visually ascertain the target tissue H through the optical clearing site X (“YES” in step S4), the procedure proceeds to the fifth step S5.

On the other hand, in the fourth step S4, in the case in which it is not possible to visually ascertain the target tissue H through the optical clearing site X (“NO” in step S4), the surgeon waits further or the optical clearing agent Y is additionally applied to the optical clearing site X. Specifically, in the case in which the waiting time is insufficient (“NO” in step S7), the surgeon waits by further (step S3). On the other hand, in the case in which a sufficient amount of time has already passed while waiting (“YES” in step S7), the applied amount of the optical clearing agent Y is determined to be insufficient, and thus, the optical clearing agent Y is applied to the optical clearing site X again (step S2), and the surgeon waits again (step S3).

Next, in the fifth step S5, while observing the target tissue H through the optical clearing site X by using the laparoscope 1, the target tissue H is treated by using surgical instruments inserted into the abdominal cavity. For example, the cystic duct B and the cystic artery are exposed by detaching the fat tissue that covers the cystic duct B and the cystic artery, the cystic duct B and the cystic artery are clipped, and the cystic duct B and the cystic artery are cut. Subsequently, the gallbladder A is removed from the abdominal cavity.

Next, in the sixth step S6, the optical clearing agent Y is washed off by washing the optical clearing site X with a washing liquid such as a physiological saline solution. In the case in which the optical clearing agent Y is injected on the inner side of the membrane F in the second step S2, it is preferable that an incision be made in the membrane F before washing. By doing so, a state in which the optical clearing agent Y and the washing liquid easily flow out from the inner side of the membrane F is achieved, and thus, it is possible to more efficiently wash off the optical clearing agent Y.

As has been described above, with this embodiment, it is possible to observe the target tissue H covered with the fat tissue G by using the laparoscope 1 by means of the simple procedure in which the optical clearing agent Y is allowed to act on the fat tissue G. By enhancing the visibility of the target tissue H in this way, there is an advantage in that the surgeon can perform treatment, such as detaching the fat tissue or the like, in a simple manner while accurately recognizing the target tissue H and the peripheral tissue thereof.

The following invention is derived from the above-described embodiment.

An aspect of the present invention is a laparoscopic surgical method including: observing the interior of the abdominal cavity by using a laparoscope and selecting an optical clearing site to be subjected to optically clearing of fat tissue that covers target tissue; allowing an optical clearing agent to act on the selected optical clearing site; checking the visibility of the target tissue through the optical clearing site on which the optical clearing agent has been allowed to act; and treating the target tissue while observing, by using the laparoscope, the target tissue in which the visibility thereof has been checked.

With this aspect, a portion of the fat tissue that covers the target tissue is selected as the optical clearing site, and the optical clearing site is made transparent by allowing the optical clearing agent to act on the optical clearing site. Next, the target tissue is treated after checking the fact that it is possible to visually ascertain the target tissue through the optical clearing site.

In this way, by clearing the fat tissue by using the optical clearing agent, a satisfactorily optical clear path is ensured between the laparoscope disposed on the outer side of the fat tissue and the target tissue positioned on the inner side of the fat tissue. In other words, a simple procedure of merely allowing the optical clearing agent to act on the fat tissue makes it possible to observe the target tissue covered with the fat tissue by using the laparoscope, and thus, it is possible to treat the target tissue in a simple manner while accurately recognizing the target tissue.

In the above-described aspect, the allowing the optical clearing agent to act may include injecting the optical clearing agent on an inner side of a membrane that covers the fat-tissue surface.

By doing so, the optical clearing agent is retained for a certain period of time in a state in which, on the inner side of the membrane, the optical clearing agent is in direct contact with the fat tissue. By doing so, it is possible to allow the optical clearing agent to more efficiently act on the fat tissue.

In the above-described aspect, the allowing the optical clearing agent to act may include piercing the membrane with a needle and the injecting optical clearing agent between the membrane and the fat-tissue surface.

By using the needle in this way, it is possible to easily control the injection position and the injected amount of the optical clearing agent. In addition, by injecting the optical clearing agent between the membrane and the fat-tissue surface, it is possible to allow the optical clearing agent to reliably act on the portion from the fat-tissue surface to the target tissue, and thus, it is possible to reliably ensure a satisfactorily optical clear path from the laparoscope disposed on the outer side of the fat tissue to the target tissue.

The above-described aspect may include waiting until the optical clearing agent permeates the fat tissue after the allowing the optical clearing agent to act.

By doing so, it is possible to allow the optical clearing agent to more reliably act on the fat tissue.

The above-described aspect may include washing the optical clearing site after the treating.

By doing so, it is possible to wash off the optical clearing site that is no longer needed.

In the above-described aspect, the checking visibility may include observing a blood vessel, which serves as the target tissue, by means of Narrow Band Imaging.

By using the NBI, with which it is possible to specifically observe the blood vessel, it is possible to accurately check the visibility of the blood vessel, which serves as the target tissue.

In the above-described aspect, the checking visibility may include observing tubular tissue, which serves as the target tissue, by using a fluorescent agent.

By using the fluorescent agent, with which it is possible to specifically observe the tubular tissue, it is possible to accurately check the visibility of the tubular tissue, which serves as the target tissue.

### REFERENCE SIGNS LIST

1 laparoscope
2 forceps
3 needle
A gallbladder
B cystic duct
C common hepatic duct
D common bile duct
E duodenum
F membrane
G fat tissue
H target tissue
X optical clearing site
Y optical clearing agent

1. A laparoscopic surgical method comprising:
observing an interior of an abdominal cavity by using a laparoscope and selecting an optical clearing site to be subjected to optically clearing of fat tissue that covers target tissue;
allowing an optical clearing agent to act on the selected optical clearing site;
checking visibility of the target tissue through the optical clearing site on which the optical clearing agent has been allowed to act; and
treating the target tissue while observing, by using the laparoscope, the target tissue in which the visibility thereof has been checked.

2. A laparoscopic surgical method according to claim 1, wherein, the allowing the optical clearing agent to act includes injecting the optical clearing agent on an inner side of a membrane that covers the fat-tissue surface.

3. A laparoscopic surgical method according to claim 2, wherein, the allowing the optical clearing agent to act includes piercing the membrane with a needle and injecting the optical clearing agent between the membrane and the fat-tissue surface.

4. A laparoscopic surgical method according to claim 1, further comprising:
after the allowing the optical clearing agent to act, waiting until the optical clearing agent permeates the fat tissue.

5. A laparoscopic surgical method according to claim 1, further comprising:
after the treating, washing the optical clearing site.

6. A laparoscopic surgical method according to claim 1, wherein, the checking visibility includes observing a blood vessel, which serves as the target tissue, by means of Narrow Band Imaging.

7. A laparoscopic surgical method according to claim 1, wherein, the checking visibility includes observing tubular tissue, which serves as the target tissue, by using a fluorescent agent.
